(19) United States
(12) Patent Application Publication
(10) Pub. No.: US 2021/0154030 A1
(43) Pub. Date: May 27, 2021

(54) Low Extensibility Strips for Prosthetic and Orthotic Applications

(71) Applicants: Aldo Laghi, Pinellas Park, FL (US); Alps South Europe S.R.O., Plzen (CZ)

(72) Inventor: Aldo Laghi, Pinellas Park, FL (US)

(21) Appl. No.: 17/094,885

(22) Filed: Nov. 11, 2020

Related U.S. Application Data
(60) Provisional application No. 62/938,483, filed on Nov. 21, 2019.
(60) Provisional application No. 62/942,388, filed on Dec. 2, 2019.

(51) Int. Cl. A61F 2/78; A61F 5/01

(52) U.S. Cl. CPC A61F 2/7812; A61F 5/0118; A61F 5/0111

(57) ABSTRACT

A low extensibility material for use in prosthetics and orthotics. The low extensibility material is adhered to the fabric portion of a prosthetic liner or orthotic liner either internally or externally and limits the longitudinal movement while allowing for transversal movement. The customizable nature of the invention allows for shaping and personalization depending on the needs of the user.

### CROSS-REFERENCE TO RELATED INVENTIONS

This application claims priority to U.S. Provisional Patent Application No. 62/938,483 filed Nov. 21, 2019 and to U.S. Provisional Patent Application No. 62/942,388 filed Dec. 2, 2019, the contents of which are incorporated by reference herein in their entirety.

### BACKGROUND OF THE INVENTION

### Field of the Invention

The described invention relates to strips to prevent the elongation of prosthetics and orthotics in certain directions. Specifically, the described invention relates to low extensibility strips which reduce elongation in the longitudinal direction when applied to prosthetics and orthotics.

### Description of the Background Art

Silicone liners have been used since the 1980s in the prosthetic industry such as those described in U.S. Pat. No. 4,923,474 granted to Klasson and Kristinsson. Other examples of such liners include U.S. Pat. No. 5,728,168 to Laghi et al., U.S. Pat. No. 5,830,237 granted to Kania, U.S. Pat. No. 5,507,834 to Laghi et al., U.S. Pat. No. 5,443,525 to Laghi et al., and U.S. Pat. No. 5,728,168 to Laghi et al. Gel and urethane liners have also been used for prosthetic and orthotic purposes and, for the most part, have a fabric covering. The fabric covering is used to reinforce the underlying material (silicone, gel, urethane) and allows for extensibility to make it easier to don and doff the liner by rolling it on and off the residual limb.

However, there exists a need for liners with limited extensibility in localized regions of the residual limb and greater extensibility in other regions of the residual limb. Liners such as those described in U.S. Pat. No. 9,216,099 to Laghi, U.S. Pat. Nos. 9,364,347, 8,394,150, 8,852,291, 8,246,694, 8,808,294, 8,226,732, 8,357,206, 6,764,631, 6,544,292, 6,454,812, 5,728,168 and 5,507,834 each describe liners which highlight such a need.

U.S. Pat. No. 6,231,617 to Fay, which is incorporated herein by reference, describes the a liner which incorporates elongate arms of a strip or ribbon shape. However, a limitation of Fay is that the arms have to radiate from the distal attachment plate or mounted to a peripheral edge of the distal attachment plate. The present invention allows for the use of low extensibility strips anywhere on or in the prosthetic/orthotic without being limited to a certain location on the device.

Generally, the distal end of locking liners require the fabric to be less extensible in the longitudinal direction or that a polymer or other solid implement be used in order to reduce pistoning of the prosthetic implement during ambulation or movement. Other regions may benefit from reduced extensibility of the fabric cover such as in the case the above the knee amputees who may exhibits unusually fleshy residual limbs.

The present invention overcomes the aforementioned inadequacies of the prior art and current need in the industry by using low extensibility fabric adhered externally to the fabric cover of the liner or integrally formed with the liner internally.

Therefore, it is an object of this invention to provide an improvement which overcomes the aforementioned inadequacies of the prior art devices and provides an improvement which is a significant contribution to the advancement of the liner art.

Another object of the invention is to provide strips or other shapes of low extensibility material which can be applied to the exterior of fabric liners.

Another object of the invention is to provide low extensibility material which, when applied to fabric liners, reduce extensibility in the longitudinal direction while maintaining extensibility in the transversal direction.

Another object of the invention is provide a fast and inexpensive way to reduce the extensibility of fabric liners in desired regions.

Another object of the invention is to provide a customizable solution for patient comfort such that a prosthetist can adhere appropriate shapes in appropriate locations in order to support or contain a region or segment of the residual limb as required by patient clinical conditions.

Another object of the invention is to contain the dynamic deformation of the residual limb during ambulation.

Another object of the invention is to provide a low extensibility material which can be cut into customer or standard shapes.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The present invention relates generally to low extensibility strips for use with prosthetic/orthotic devices. Specifically, the present invention relates to low extensibility material for use in prosthetics and orthotics which is adhered to the fabric portion of a prosthetic liner or orthotic liner either internally or externally and limits the longitudinal movement while allowing for transversal movement. The customizable nature of the invention allows for shaping and personalization depending on the needs of the user

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a cross-sectional view of a liner incorporating the low extensibility material internally;

FIG. 2 is a top view of the low extensibility material in one preferred shape;

FIG. 3 is a top view of the low extensibility material in a second preferred shape;

FIG. 4 is a top view of the low extensibility material in a third preferred shape and configuration;

FIG. 5 is a top view of the low extensibility material in a second preferred configuration;

FIG. 6 is a cross-sectional view of a liner with an alternative arrangement of low extensibility material placed either internally or externally;

FIG. 7 is a cross-sectional view with an alternative arrangement of low extensibility material placed either internally or externally;

FIG. 8 is a perspective view of a wrist orthotic with the low extensibility material adhered externally;

FIG. 9 is a perspective view of a back orthotic with the low extensibility material adhered externally;

FIG. 10A is a perspective view of an ankle orthotic with the low extensibility material adhered externally;

FIG. 10B is a cross-sectional view of the ankle orthotic shown in FIG. 10A;

FIG. 11 is a perspective view of a knee orthotic with the low extensibility material adhered externally; and

FIG. 12 is a perspective view of an elbow orthotic with the low extensibility material adhered externally.

Similar reference numerals refer to similar parts throughout the several views of the drawings.

### DETAILED DESCRIPTION OF THE PREFERRED EMBODIMENT

The following description is of the best mode presently contemplated for carrying out the invention. This description is not to be taken in a limiting sense, but is made merely for the purpose of describing one or more preferred embodiments of the invention. The scope of the invention should be determined with reference to the claims.

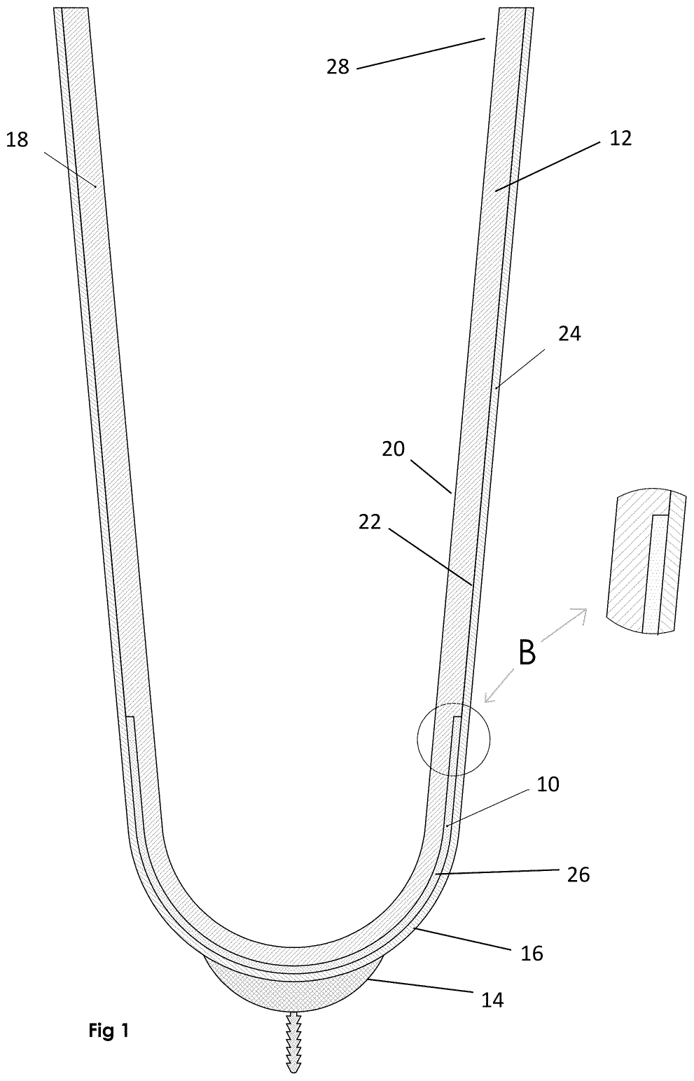

The present invention relates to a low extensible material 10 for use with prosthetic or orthotic devices. As shown in FIG. 1, this low extensibility material 10 can be used with a prosthetic liner 12 having a distal attachment plate 14 at a distal end 16. The low extensibility material 10 for use with a prosthetic or orthotic assembly preferably comprises a strip of material 26 preferably made of strips of silk, fiberglass cloth, and other non-elastic materials like carbon fiber and thermoplastics. The prosthetic liner 12 comprises an elastomer layer 18 having a limb face 20 and a fabric face 22 wherein the fabric face 22 of the elastomer layer 18 is adhered to the external fabric 24 of the prosthetic liner 12. In relation to the prosthetic liner 12, the low extensibility material 10 can be adhered to the fabric 24 either internally or externally. If internal placement is desired, the low extensibility material 10 is placed between the elastomer layer 18 and the fabric 24 in the desired region and adhered only to the fabric 24 as shown in the exploded view Part B. Preferably, the low extensibility material 10 is placed at the distal end 16 of the prosthetic liner 12 and extends towards the proximal end 28 of the prosthetic liner 12. The low extensibility material 10 preferably extends upward towards the proximal end 28 of the prosthetic liner 12, stopping at least an inch from the proximal end 12, when placed at the distal end 16. Similarly, the width of the low extensibility material 10 is between 0.5-4 inches such that it can be placed in a variety of locations and positions.

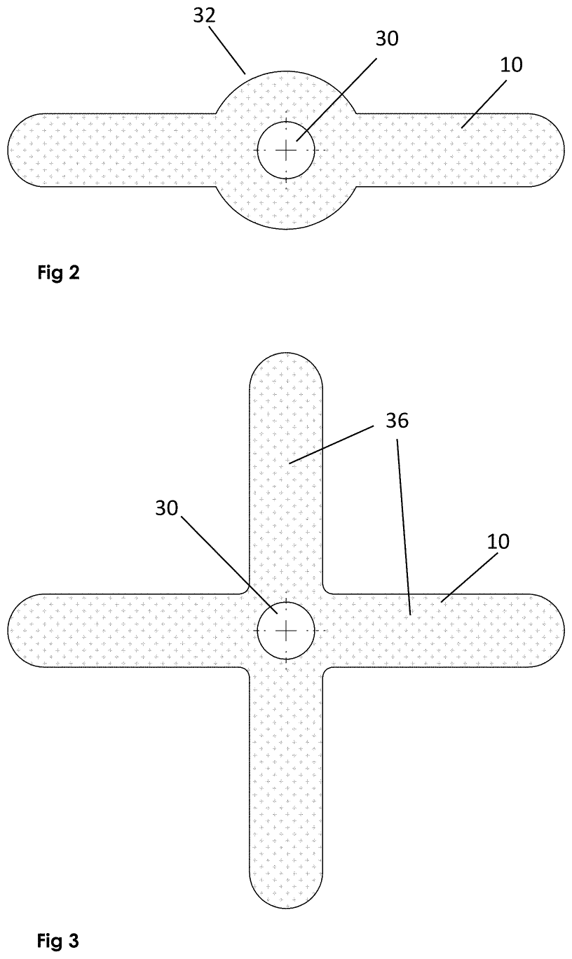

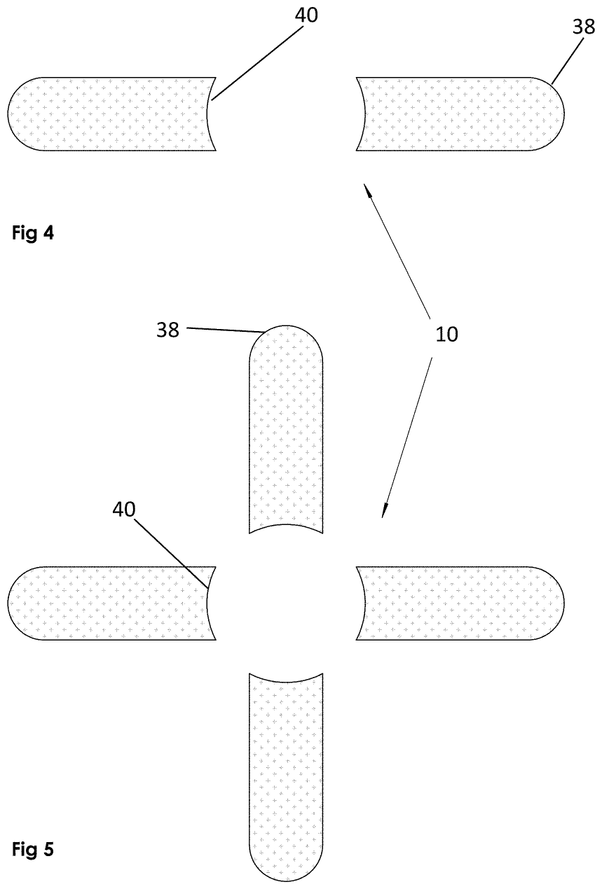

FIGS. 2-7 show the low extensibility material 10 is a variety of configurations. FIG. 2 shows the low extensibility material 10 as a strip of material having a central aperture 30 with a bulged mid-region 32. The locking pin 34, as shown in FIG. 1, can be fed through the aperture 30 if the low extensibility material 10 is adhered externally to the fabric 24. As shown in FIG. 3, the low extensibility material 10 can also be configured to have multiple arms 36 extending radially outward from the central aperture 30. As shown in FIGS. 4 and 5, the aperture 30 is optional. The low extensibility material 10 can be shaped such that a distal strip end 38 is convex while a proximal strip end 40 is concave. This allows for the low extensibility material 10 to be placed around the distal attachment plate 14 if desired or around the user's joint without impinging on joint movement.

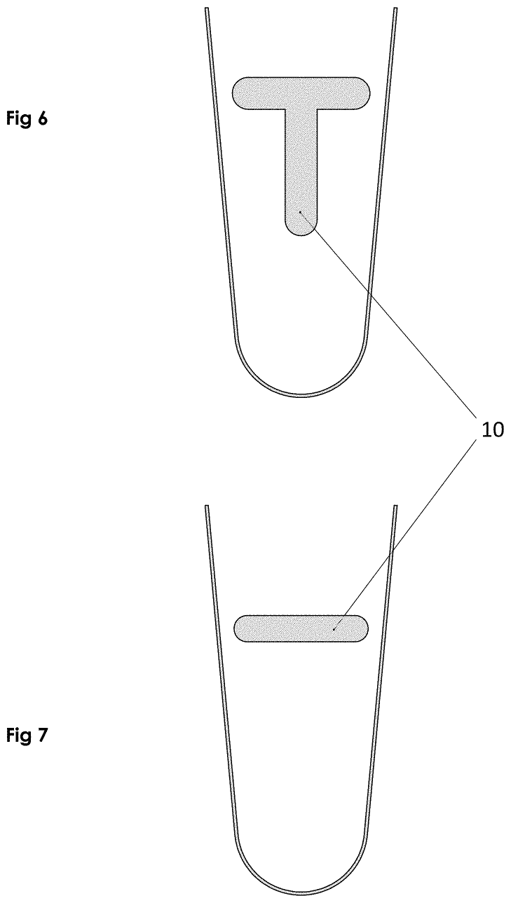

As seen in FIGS. 6 and 7, the low extensibility material 10 can be placed in a variety of configurations according to the needs of the user. The T-shape and single strip configuration shown are not the only configurations that would be helpful to a user and any configuration determined by a medical professional to be medically helpful can be used.

Use of low extensibility material 10 as described herein has been tested and conclusively shows that use of the low extensibility material 10 limits the longitudinal stretch of the liner 12 while maintaining transversal stretch. In the first table, transversal stretch was tested:

| | Reinforced | | | | | Not Reinforced | | | |
|---|---|---|---|---|---|---|---|---|---|
| | before | after | change | % | | before | after | change | % |
| 1 | 13 | 21 | 8 | 61.53846 | 1 | 13 | 20.375 | 7.375 | 56.73077 |
| 2 | 13 | 22 | 9 | 69.23077 | 2 | 13 | 21.5 | 8.5 | 65.38462 |
| 3 | 13 | 20.125 | 7.125 | 54.80769 | 3 | 13 | 22 | 9 | 69.23077 |

| | | | |
|---|---|---|---|
| Average | 61.85897 | Average | 63.78205 |

As seen in the table above, “before” refers to the liner in a static state, meaning no weight was applied, and “after” refers to the liner at failure (i.e. until the strip tore). The number measured is circumference in inches. As can be seen from the test data, the transversal stretch was essentially the same whether the liner was reinforced with the low extensibility material 10 or not.

A similar test was performed testing the longitudinal stretch:

| | Reinforced | | | | | Not Reinforced | | | |
|---|---|---|---|---|---|---|---|---|---|
| | before | after | change | % | | before | after | change | % |
| 1 | 4.25 | 4.5 | 0.25 | 5.882353 | 1 | 4 | 4.75 | 0.75 | 18.75 |
| 2 | 4.25 | 4.5 | 0.25 | 5.882353 | 2 | 4.25 | 4.75 | 0.5 | 11.76471 |
| 3 | 4.5 | 4.75 | 0.25 | 5.555556 | 3 | 4.25 | 4.625 | 0.375 | 8.823529 |

| | | | |
|---|---|---|---|
| Average | 5.77342 | Average | 13.11275 |

As can be seen from this test, the longitudinal stretch was reduced by more than half when the low extensibility material 10 was used.

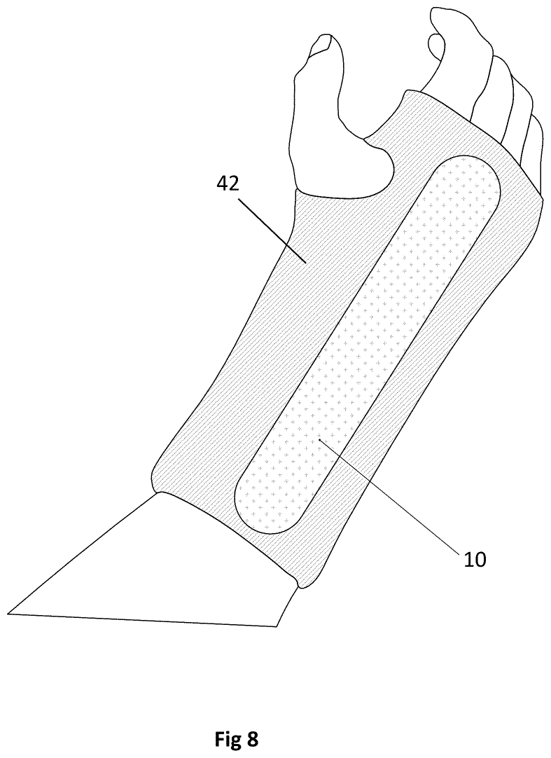

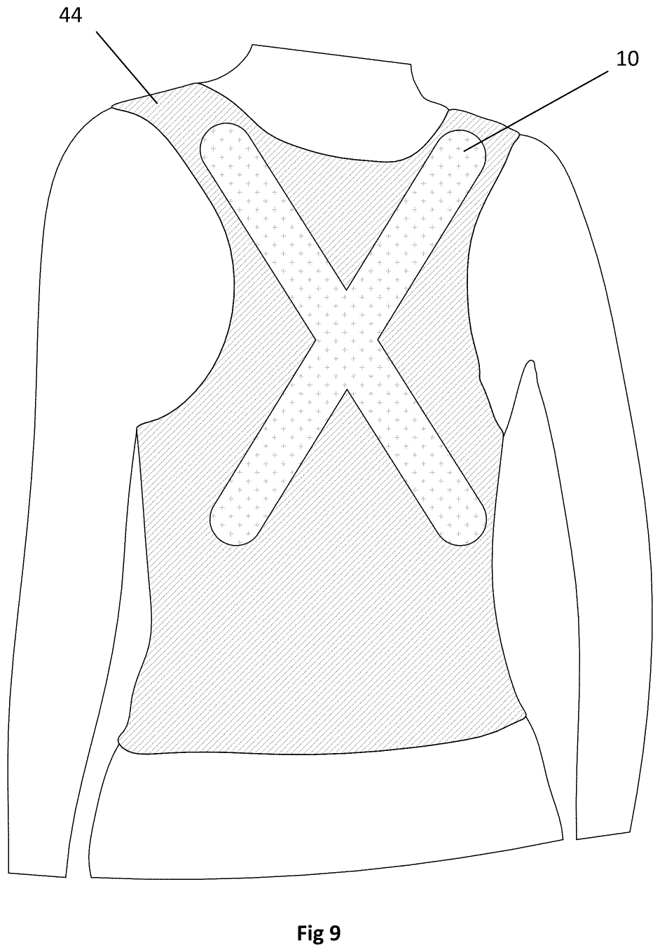

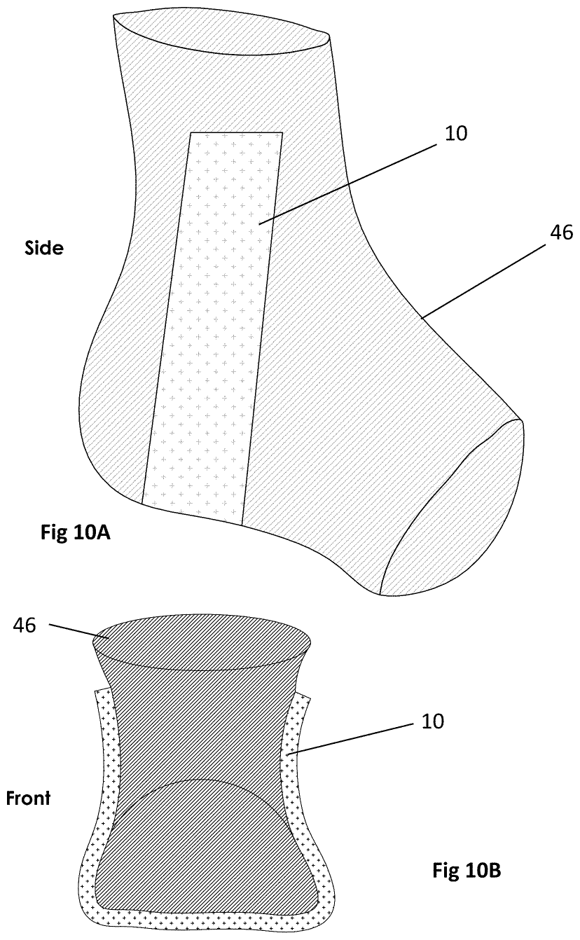

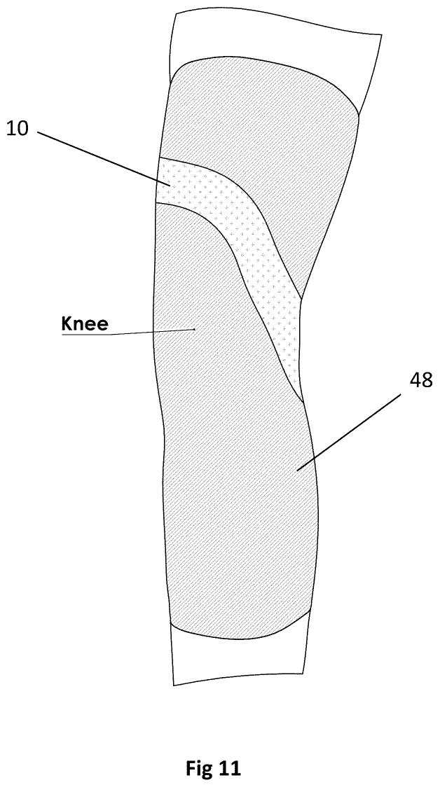

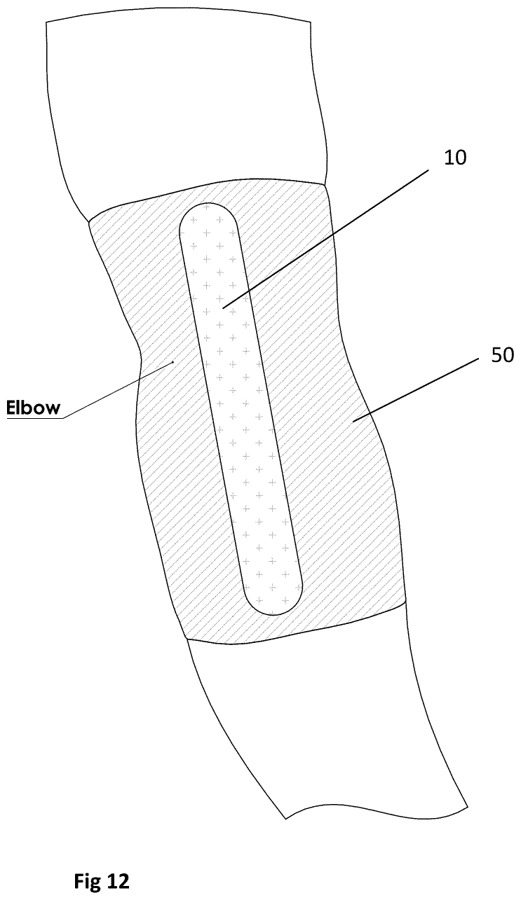

The low extensibility material 10 can also be used with orthotics which is useful to limit joint flexion both as a therapeutic aid in the case of injuries or as a means of injury prevention. As seen in FIG. 8, the low extensibility material 10 works with a wrist brace 42 and can be adhered externally or internally as described above. Similarly, as shown in FIG. 9, the low extensibility material 10 can be integrated with a back brace orthotic 44. Likewise, the low extensibility material 10 can be used with an ankle orthotic 46, as shown in FIGS. 10A and 10B, where the low extensibility material 10 preferably has a thickness to prevent ankle movement. FIG. 11 depicts the use of the low extensibility material 10 with a knee orthotic 48 and FIG. 12 depicts the use of the low extensibility material 10 on an elbow orthotic 50. In all configurations when in use with an orthotic, the low extensibility material 10 can be in a strip or in a specific shape, as determined by an orthotic physician, and be adhered to the fabric 24 either internally or externally.

When the low extensibility material 10 is desired to be used internally, the low extensibility material 10 is first adhered to the fabric 24 using conventional means and then the combination is placed in a mold wherein the gel, silicon, or urethane is molded over, creating a composite. Alternatively, exterior use is performed by adhering the low extensibility material 10 onto the outside of the fabric 24 in the desired location.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A prosthetic apparatus, comprising:
a prosthetic liner having a closed distal end and an open proximal end, the prosthetic liner further comprising an elastomer layer and a fabric layer;
a single-piece low extensibility material in between said elastomer layer and said fabric layer extending up to six inches from the open proximal end and having a width between 0.5 and 4 inches, the single-piece low extensibility material further comprising a central aperture; and
wherein longitudinal stretch of the prosthetic liner is reduced by more than 50% as compared to a non-reinforced prosthetic liner.

2. A prosthetic apparatus, comprising:
a prosthetic liner having a closed distal end and an open proximal end, the prosthetic liner further comprising an elastomer layer and a fabric layer having an external face;
at least one strip of a low extensibility material affixed to the external face of the fabric layer within six inches of the proximal end creating a reduced stretch area, the low extensibility material having a width between 0.5 and 4 inches; and
wherein the reduced stretch area has a reduced longitudinal stretch of more than 50% as compared to a non-reinforced liner area.

3. The prosthetic apparatus of claim 2 wherein at least two strips of low extensibility are affixed to the external face of the fabric layer in a T-shape.

4. A low extensibility apparatus, comprising:
an orthotic device comprising an elastomer layer and a fabric layer having an extern face;
at least one strip of a low extensibility material placed on the external face of the fabric layer within six inches of the proximal end creating a reduced stretch area, the low extensibility material having a width between 0.5 and 4 inches; and
wherein the reduced stretch area has a reduced longitudinal stretch of more than 50% as compared to a non-reinforced liner area.

5. The low extensibility apparatus of claim 3 wherein the orthotic device is a wrist brace.

6. The low extensibility apparatus of claim 3 wherein the orthotic device is a back brace.

7. The low extensibility apparatus of claim 3 wherein the orthotic device is an ankle brace.
